(19) United States
(12) Patent Application Publication
(10) Pub. No.: US 2026/0090960 A1
(43) Pub. Date: Apr. 2, 2026

(54) METHOD OF VITAL PULP THERAPY USING BIOACTIVE COMPOSITES

(71) Applicant: IMAM ABDULRAHMAN BIN FAISAL UNIVERSITY, Dammam (SA)

(72) Inventors: Mohammed ALGHARNI, Dammam (SA); Omar OMAR, Dammam (SA); Fatimah DAHLAWI, Dammam (SA); Jehan ALHUMAID, Dammam (SA)

(73) Assignee: IMAM ABDULRAHMAN BIN FAISAL UNIVERSITY, Dammam (SA)

(21) Appl. No.: 18/901,829

(22) Filed: Sep. 30, 2024

(51) Int. Cl. A61K 6/849; A61K 6/84; A61K 31/739

(52) U.S. Cl. CPC A61K 6/849; A61K 6/84; A61K 31/739

(57) ABSTRACT

A method of vital pulp therapy with a bioactive capping material. The method includes drilling into a pulp of a tooth having decay to form a hole, then excavating infected pulp from the hole. The bioactive capping material is prepared by mixing glass ionomer cement (GIC) and potassium nitrate (KNO₃) with a solvent. The method further includes covering healthy pulp in the tooth with the bioactive capping material and curing the bioactive capping material to form a cured capping material. The bioactive capping material includes KNO₃ in an amount of 1 to 10 percent by weight (wt. %) based on the weight of the capping material.

### BACKGROUND

### Technical Field

The present disclosure is directed to a method of vital pulp therapy and particularly, towards a pulp therapy using glass ionomer cement (GIC) and potassium nitrate (KNO₃) bioactive capping material.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Dental pulp is a type of connective tissue found within the dentine and enamel of the teeth. When exposed to damage, such as caries or tooth fracture, dental pulp is at risk of infection, which can lead to pain, necrosis, and infection of the jaw bone and surrounding tissues. A root canal, consisting of a complete pulpectomy (full removal of the dental pulp), is the most common treatment for infected pulp. A complete pulpectomy ensures the removal of all dental pulp, whether infected or not. Root canals, however, can be at risk of structural failure due to the high forces exerted on teeth during mastication (i.e., chewing). Teeth treated with root canal treatment may also be at risk of loss of sensation, potentially leading to more, or worse, caries, rejection of the foreign material, reinfection, and occurrence of apical periodontitis. Further, it is nearly, if not impossible, to completely disinfect the inside of a tooth during root canal treatment due to the complex anatomical canals of the tuth and surrounding microscopic tubules, which contain proteins and other tissues acquired from the now-absent pulp. Both the anatomical canals and microxcopic tubules can be traps for harmful bacteria. Once present in the tooth receiving the root canal, the bacteria can then freely to enter a patient's general circulation via the root tips. Health problems linked to lingering infection in root canal teeth include autoimmune and musculoskeletal diseases, irritable bowel diseases, fibromyalgia, and some cancers. Accordingly, there is a need for an alternative treatment strategy that poses less risk for future complications.

Vital pulp therapy (VPT) is a potential alternative to root canal treatment. VPT is a restorative dental procedure that aims to treat teeth with compromised dental pulp without the full removal or excavation of all healthy pulp tissue. VPT may include indirect or direct pulp capping (i.e., placement of a protective material over the pulp) and a partial pulpotomy (i.e., removal of part of the dental pulp). A partial pulpotomy ensures that only the infected dental pulp is removed, allowing only the healthy dental pulp to remain. By maintaining the healthy portion of the dental pulp, VPT aids in retaining the tooth's natural integrity and strength, which may be comprosmised in other treatment strategies such as root canal treatment. Dressings used in VPT can include resin modified glass ionomers, adhesive resins, calcium hydroxide (CaOH), mineral trioxide aggregates (MTA), and bioceramics. These materials are designed to interact with the pulp tissue to promote healing, reduce inflammation, and encourage the formation of reparative dentin. Presently, MTA is the preferred material for VPT. However, MTA is known for its difficult handling properties. It can be challenging to manipulate and place accurately, particularly in small or complex cavities. Further, MTA has a high initial pH, which can irritate the surrounding tissues immediately after placement. The setting reaction in calcium silicate materials, such as MTA, involves hydration, producing calcium hydroxide, and releasing calcium and hydroxyl ions. This increase in pH causes a rise in the pH of the underlying tissue, resulting in a thin necrotic layer between the remaining vital tissue and the pulp-capping agent. Consequently, a challenge of VPT is the selection of the dressings and the unpredictability of the pulp's healing response. Thus, there exists a need to improve the efficacy of the dressings utilized in VPT.

Accordingly, one object of the present disclosure is to produce a bioactive dental capping material that reduces the inflammatory response and enhances the secretion of regenerative signals around the damaged tooth, leading to the promotion of healing, the reduction of inflammation, and the formation of reparative dentin

### SUMMARY

In an exemplary embodiment, a method of vital pulp therapy using a bioactive capping material is described. The method comprises drilling into a pulp of a tooth having decay to form a hole, then excavating infected pulp from the hole. The method further comprises mixing a glass ionomer cement (GIC) and potassium nitrate (KNO₃) with a solvent to form a bioactive capping material. The method further comprises covering the remaining healthy pulp in the tooth with the bioactive capping material and curing the bioactive capping material to form a cured capping material. The bioactive capping material comprises KNO₃ in an amount of 1 to 10 percent by weight (wt. %) based on the weight of the capping material.

In some embodiments, the method comprises combination of the ratio of the solvent to a combined amount of glass ionomer cement (GIC) and potassium nitrate (KNO₃) in ratio of 1:3 to 3:1.

In some embodiments, a solvent is selected from the group consisting of water, ethanol, methanol, and acetone.

In some embodiments, the solvent is water.

In some embodiments, the cured capping material has a compressive strength of at least 450 newtons per square centimeter (N/cm²).

In some embodiments, the cured capping material has a microhardness of no more than 40 HV.

In some embodiments, the KNO₃ is present in an amount of 5 to 10 wt. % based on the weight of the capping material.

In some embodiments, the bioactive capping material has a pH of 6 to 7.

In some embodiments, a cured capping material has a compressive strength of at least 500 N/cm².

In some embodiments, the cured capping material has a microhardness of no more than 35 Vickers hardness (HV).

In some embodiments, the bioactive capping material has a pH of 6.5.

In another embodiment, a method for treating inflammation near a tooth is described. The method comprises drilling into a pulp of a tooth of a patient to form a hole, then excavating infected pulp from the hole, wherein the tooth has a cavity breaching at least a surface of the pulp and administering a bioactive dental composite comprising a glass ionomer cement (GIC) and potassium nitrate to at least one surface of remaining healthy pulp of the tooth of a patient. The KNO₃ is present in an amount of 1 to 10 weight by percent (wt. %) based on the weight of the dental composite. The dental composite aids in releasing a substance that encourages tissue regeneration while reducing the release of substances that cause inflammation.

In some embodiments, a regenerative marker has a release level of at least 40 picograms per milliliter (pg/mL).

In some embodiments, the regenerative marker is at least one selected from the group consisting of growth differentiation factor (GDF), inhibin A (INHA), myostatin (MSTN), and transforming growth factor (TGF-β).

In some embodiments, the inflammatory marker has a release level of at least 25 pg/mL.

In some embodiments, the inflammatory marker is at least one selected from the group consisting of C-reactive protein (CRP), serum amyloid A, procalcitonin, cytokines, TNFα, and interleukins 6 (IL-6).

In some embodiments, a bioactive dental composite releases at least one of nitrate, potassium, and fluoride on the surface of the tooth.

In some embodiments, the bioactive dental composite has an average potassium release level of at least 1000 parts per million (ppm).

In some embodiments, the bioactive dental composite has an average nitrate release level of at least 50 ppm.

In some embodiments, the bioactive dental composite has an average fluoride release level of at least 4 ppm.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a schematic flow chart of a method of vital pulp therapy using bioactive materials, according to certain embodiments.

FIG. 2A is a column graph depicting secretion levels of inflammatory (IL-6) indicating statistically significant differences between the experimental groups with and without LPS by asterisks (P<0.05*, P<0.0001***), according to certain embodiments.

FIG. 2B is a column graph depicting secretion levels of regenerative (TGF-β) indicating statistically significant differences between the experimental groups with and without LPS by asterisks (P<0.05*, P<0.0001***).

FIG. 3A is an experimental procedure for buffy coat mixed and diluted with equal volume of Hank's Balanced Salt Solution (HBSS) and layered on the top of Ficoll-Paque PREMIUM, according to certain embodiments.

FIG. 3B shows a buffy coat mixture centrifuged and the layer of mononuclear cells ready for aspiration, according to certain embodiments.

FIG. 3C shows fluorescent staining of cells after isolation using Actin staining kit (ActinRed™ 555) for cell body along DAPI stain to reveal the nuclei, according to certain embodiments.

FIG. 3D, FIG. 3E, and FIG. 3F show trans-well system used with disk sample arranged in the 24-well plate in 1 mm seeded monocytes in culture media and seeded monocytes after 24 hours under microscope before disk placement, according to certain embodiments.

FIG. 3G shows microscopic image of 1 mm seeded monocytes in culture media after 24 hours before disk placement without LPS, according to certain embodiments.

FIG. 3H shows microscopic image of 1 mm seeded monocytes in culture media after 24 hours before disk placement with LPS, according to certain embodiments.

FIG. 3I shows summary of the experimental and analytical procedures, according to certain embodiments.

FIG. 4A shows a column graph of cell counting analysis of total number of attached cells (cell/ml) after 1 day of culture without (control) or with indirect exposure to the different materials, the absence or presence of LPS stimulation, according to certain embodiments.

FIG. 4B shows a column graph of cell counting analysis of total number of detached cells (cell/ml) after 1 day of culture without (control) or with indirect exposure to the different materials, the absence or presence of LPS stimulation, according to certain embodiments.

FIG. 4C shows a column graph of cell counting analysis of total number of viable cells or live cells (cell/ml) after 1 day of culture without (control) or with indirect exposure to the different materials, the absence or presence of LPS stimulation, according to certain embodiments.

FIG. 4D shows a column graph of cell counting analysis of total number of dead cells (cell/ml) after 1 day of culture without (control) or with indirect exposure to the different materials, the absence or presence of LPS stimulation, according to certain embodiments.

FIG. 5A is a column graph depicting direct disk PH (material discs), according to certain embodiments.

FIG. 5B is a graph depicting PH change over 4 days in culture media, according to certain embodiments.

FIG. 6A is a column graph depicting potassium release analysis of disks Ctrl=control; MTA=mineral trioxide aggregate; GIC=glass ionomer cement; GIC-5=glass ionomer cement with 5% substitution with potassium nitrate; GIC-10=glass ionomer cement with 10% substitution with potassium nitrate, according to certain embodiments.

FIG. 6B is a column graph depicting nitrate release analysis of disks Ctrl=control; MTA=mineral trioxide aggregate; GIC=glass ionomer cement; GIC-5=glass ionomer cement with 5% substitution with potassium nitrate; GIC-10=glass ionomer cement with 10% substitution with potassium nitrate, according to certain embodiments.

FIG. 6C is a column graph depicting fluoride release analysis of disks Ctrl=control; MTA=mineral trioxide aggregate; GIC=glass ionomer cement; GIC-5=glass ionomer cement with 5% substitution with potassium nitrate; GIC-10=glass ionomer cement with 10% substitution with potassium nitrate, according to certain embodiments.

FIG. 7 is a column graph depicting compressive strength test indicating statistically significant differences between the experimental groups with and without LPS by asterisks (P<0.05*), according to certain embodiments.

FIG. 8 is a column graph depicting microhardness test indicating statistically significant differences between the experimental groups with and without LPS by asterisks (P<0.05*), according to certain embodiments.

FIG. 9A is a column graph depicting level of cytotoxicity Lactate Dehydrogenase (LDH) data by representing statistically significant differences between the experimental groups with and without LPS using asterisks (P<0.05*, P<0.0001***), according to certain embodiments.

FIG. 9B is a column graph depicting number of attached viable cells indicating statistically significant differences between the experimental groups with and without LPS by asterisks (P<0.001**), according to certain embodiments.

### DETAILED DESCRIPTION

When describing the present disclosure, the terms used are to be construed in accordance with the following definitions, unless a context dictates otherwise. Embodiments of the present invention will now be described more fully hereinafter with reference to the accompanying drawings wherever applicable, in that some, but not all embodiments of the disclosure are shown.

In the drawings, reference numerals designate identical or corresponding parts throughout the several views. Further, as used herein, the words ‘a,’ ‘an,’ and the like generally carry a meaning of ‘one or more,’ unless stated otherwise.

Furthermore, the terms ‘approximately,’ ‘approximate,’ ‘about,’ and similar terms generally refer to ranges that include the identified value within a margin of 20%, 10%, or preferably 5%, and any values therebetween.

As used herein, the term “endodontic applications” refers to different procedures and treatments related to preserving tooth function and health while addressing infections or damage, mainly aimed at the dental pulp and root canals.

As used herein, the term ‘vital pulp therapy’ refers to a dental treatment intended for preserving the health of the tooth's pulp when it is inflamed or infected but still alive. Vital pulp therapy involves procedures such as pulp capping and pulpotomies, which aim to protect and maintain the pulp while avoiding more invasive treatments.

As used herein, the term “pulp tissue” refers to the innermost part of a tooth. The pulp tissue comprises nerves, blood vessels, and connective tissue. The pulp assists tooth vitality, sensory functions, and dentin formation. The dental pulp is an essential constituent to the tooth's vascularity. The pulp further comprises the tooth's stem cell population which allows the pulp to fight infection, support odontoblast-mediated dentin formation and repair, and provide nourishment and innervation to the tooth. The pulp further aids in the generation of new odontoblasts to repair dentin after the occurrence of infection or trauma.

As used herein, the term “tooth decay” refers to the gradual breakdown of tooth enamel and dentin in a patient, as instigated by acids created by bacteria in plaque. This process may result in cavities, structural damage, and potential infection.

As used herein, the term “dental cavity” or “cavity” refers to structural damage in a tooth of a patient caused by tooth decay. There are three types of cavities, referred to as smooth surface, root, and pit/fissure cavities. Smooth surface cavities occur on the smooth sides of the teeth while root cavities occur on the surface over the roots of the teeth. Pit/fissure cavities occur on the chewing surface of the teeth. Left untreated, cavities may breach the enamel and dentin of the tooth, before infecting the dental pulp.

As used herein, the term ‘desensitizing agent’ refers to a substance used for reducing or removing pain and sensitivity in a specific area, often by soothing nerve endings or blocking pain signals.

As used herein, the term “Vickers Hardness” is a measure of the hardness of a material, assessed from the size of an impression produced under load using a pyramid-shaped diamond indenter. The unit of hardness given by the Vickers Hardness test is called the Vickers Pyramid Number (HV).

As used herein, the term “microhardness testing” refers to a method for measuring the surface of small parts or areas. It can also be used for measuring individual microstructures or the depth of case hardening by making a series of indentations and creating a profile of the change in hardness.

As used herein, the term “glass ionomer cement (GIC)” refers to a type of restorative and filling material that chemically bonds to the structure of a tooth through ionic bonding.

As used herein, the term “bioactive capping material” refers to a dental substance that may be applied to the exposed pulp or dentin to encourage the tooth's natural ability to heal and mend itself. This process frequently involves encouraging the production of new dentin.

As used herein, the term “compressive strength” refers to the ability of a material to bear axial loads that tend to reduce its size. This capacity is commonly determined by the greatest compressive stress a material can sustain before failing.

As used herein, the term “bioactive dental composite” refers to a restorative substance created to positively interact with the nearby tooth structure by encouraging remineralization and improving the biological reaction, typically by emitting helpful ions such as fluoride.

As used herein, the term “curing” refers to a process of hardening or setting material like dental resins or adhesives through the use of light, heat, or chemicals.

As used herein, the term “regenerative biomarker” refers to a biological marker or material utilized for evaluating or tracking tissue regeneration and repair progress.

As used herein, the term “inflammatory marker” refers to a biological substance, often found in blood or tissues that indicates the presence or extent of inflammation in the body.

As used herein, the term “cytotoxic effect” refers to the ability of a substance or treatment to cause damage to or kill cells.

A weight percent of a component, unless specifically stated to the contrary, is based on the total weight of the formulation or composition in which the component is included. For example, if a particular element or component in a composition or article is said to have 5 wt. %, it is understood that this percentage is in relation to a total compositional percentage of 100%.

The present disclosure is intended to include all hydration states of a given compound or formula, unless otherwise noted or when heating a material.

Aspects of the present disclosure are directed toward effects of a method of vital pulp therapy utilizing a bioactive capping material of KNO₃ and GIC. The inclusion of 5 wt. % KNO₃ to GIC reduces the inflammatory response and enhances the secretion of regenerative signals to promote tooth healing.

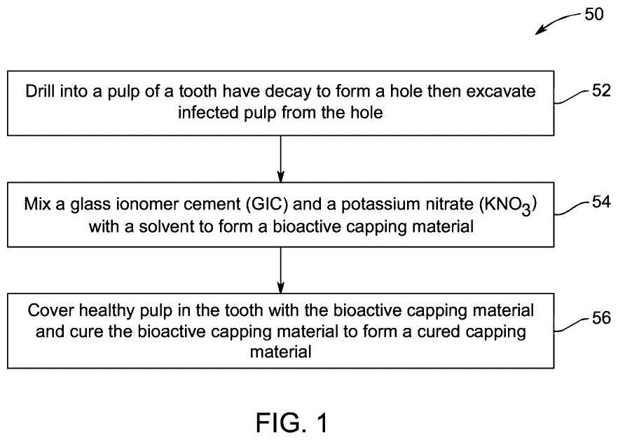

FIG. 1 illustrates a flow chart of a method 50 of vital pulp therapy with the bioactive capping material comprising KNO₃ and GIC. The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, the method 50 comprises drilling into a pulp of a tooth having a dental cavity breaching the pulp, to form a hole and then excavating infected pulp from the hole. As the drilling is performed, the tooth is undergoing lipopolysaccharide (LPS) stimulation which activates the immune system by interacting with receptors on cells and inducing the production of inflammatory cytokines. The infected or damaged pulp is removed using tools such as endodontic excavators, endodontic files, and reamers. The canals are cleaned and disinfected to prepare for sealing. In one embodiment, only the infected or damaged pulp is removed. In one embodiment, the healthy pulp remains in the tooth.

At step 54, the method 50 comprises mixing a GIC and KNO₃ with a solvent to form a bioactive capping material. GIC is a dental biocompatible material able chemically bond to the structure of a tooth. Specifically, GIC chemically bonds (ionic bonds) to the tooth's enamel and dentin. GIC bonds better to enamel than dentin because of the higher inorganic content in enamel. The mechanism of GIC adhesion to the tooth's inorganic structure involves a chelation reaction between the carboxyl groups of the polyacrylic acid and the calcium in the hydroxyapatite crystals of the tooth. GIC may offer a barrier of protection to the tooth, a low solubility, and strong wear resistance. GIC may also support remineralization through the release of fluoride into the surrounding area.

Potassium ions may interfere with the nerve signal transmission within the tooth, allowing for the management of a tooth's inflammation, damage, and/or pain. The pulpal connective tissues may be allowed to repair as a result of the reduction of the inflammatory response that arises when connective tissues are isolated. Reducing the inflammation in the tooth may generate a more favorable environment for the connective tissues, making conditions less harmful. Potassium-containing agents may aid in restoring the fluid balance and electrical charge in the pulp tissues, thus healing the pulp and preventing further tooth decay. Potassium-containing agents may also be used as a desensitizing agent, thus aiding in the management of pain and sensitivity in an inflamed or damaged tooth. In some embodiments, the potassium-containing agent in the bioactive capping material may be potassium nitrate, potassium bicarbonate, potassium bromide, potassium phosphate, potassium alum, potassium sulfate, potassium chlorate, potassium fluoride, and mixtures thereof. In a preferred embodiment, the potassium-containing agent is potassium nitrate (KNO₃).

In some embodiments, the KNO₃ is present in an amount of 1 to 10 wt. %, preferably 2 to 9 wt. %, preferably 3 to 8 wt. %, preferably 4 to 7 wt. %, and preferably 5 to 6 wt. % based on the weight of the capping material. In some embodiments, the KNO₃ is present in an amount of 5 to 10 wt. %, preferably 5 to 9 wt. %, preferably 5 to 8 wt. %, preferably 5 to 7 wt. %, most preferably 5 to 6 wt. % based on the weight of the capping material. A ratio of the solvent to a combined amount of GIC and KNO₃ is 1:5 to 5:1, preferably 1:4 to 4:1, preferably 1:3 to 3:1, preferably 1:2 to 2:1, preferably 1:2 to 1:1. In a preferred embodiment, the ratio of the solvent to a combined amount of GIC and KNO₃ is 1:2.

In some embodiments, the solvent is selected from the group consisting of water, ethanol, methanol, and acetone. In a preferred embodiment, the solvent is water. The water may be tap water, distilled water, bi-distilled water, deionized water, deionized distilled water, reverse osmosis water, and/or some other water. In a preferred embodiment, the solvent is distilled water.

At step 56, the method 50 comprises covering remaining healthy pulp in the tooth with the bioactive capping material and curing the bioactive capping material to form a cured capping material. The bioactive capping material supports tooth healing and regeneration and protects the tooth from potential damage by releasing fluoride to the surrounding area, protecting the remaining healthy pulp, inhibiting the release of inflammatory markers, and promoting the release of regenerative signals. The bioactive capping material is then subjected to a particular curing technique, which hardens it into a durable layer of protection aiding the long-standing health of the tooth. The bioactive capping material may be cured by any known method such as by ultraviolet curing, thermal curing, and the like

In some embodiments, the bioactive capping material has a pH of 6 to 7, preferably 6.05 to 6.95, preferably 6.10 to 6.90, preferably 6.15 to 6.85, preferably 6.20 to 6.80, preferably 6.25 to 6.75, preferably 6.30 to 6.70, preferably 6.35 to 6.65, preferably 6.40 to 6.60, preferably 6.45 to 6.55, most preferably 6.50 to 6.55. In a preferred embodiment, the bioactive capping material has a pH of 6.5. This pH level creates an ideal environment for tooth healing and protection by ensuring the material is biocompatible and supports the pulp health.

In some embodiments, the cured capping material has a compressive strength of at least 450 Newton per square centimeter (N/cm²), preferably at least 455 N/cm², preferably at least 460 N/cm², preferably at least 465 N/cm², preferably at least 470 N/cm², preferably at least 475 N/cm², preferably at least 480 N/cm², preferably at least 485 N/cm², preferably at least 490 N/cm², preferably at least 500 N/cm², preferably at least 505 N/cm², preferably at least 510 N/cm², most preferably at least 515 N/cm². In some embodiments, the cured capping material has a compressive strength of at least 520 N/cm². In some embodiments, the cured capping material has a microhardness of at least 25 Vickers Hardness (HV), preferably at least 30 HV, preferably at least 35 HV, preferably at least 40 HV, preferably at least 45 HV. In some embodiments, the cured capping material has a microhardness of at least 50 HV.

A method of treating inflammation near a tooth is described. The method comprises drilling into a pulp of a tooth of a patient to form a hole, then excavating infected pulp from the hole, wherein the tooth has a cavity breaching at least a surface of the pulp and administering a bioactive dental composite comprising a glass ionomer cement (GIC) and potassium nitrate to at least one surface of remaining healthy pulp of the tooth of a patient. The dental composite promotes the release of a regenerative marker and inhibits the release of an inflammatory marker. Inflammation usually involves the release of cytokines and other factors that can harm tissues and hamper healing.

In some embodiments, the regenerative marker is at least one selected from the group consisting of growth differentiation factor (GDF), inhibin A (INHA), myostatin (MSTN), and transforming growth factor (TGF-β). In a preferred embodiment, the regenerative marker is TGF-β. In some embodiments, the regenerative marker has a release level of at least 40 picograms per milliliter (pg/mL), preferably at least 42 pg/mL, preferably at least 44 pg/mL, preferably at least 46 pg/mL, preferably at least 48 pg/mL, preferably at least 50 pg/mL, preferably at least 52 pg/mL, preferably at least 54 pg/mL, preferably at least 56 pg/mL, preferably at least 58 pg/mL, preferably at least 60 pg/mL, preferably at least 62 pg/mL, preferably at least 64 pg/mL, preferably at least 66 pg/mL, preferably at least 68 pg/mL, preferably at least 70 pg/mL, preferably at least 72 pg/mL, preferably at least 74 pg/mL, preferably at least 76 pg/mL, preferably at least 78 pg/mL, preferably at least 80 pg/mL, preferably at least 82 pg/mL, preferably at least 84 pg/mL, preferably at least 86 pg/mL, preferably at least 90 pg/mL, preferably at least 92 pg/mL, preferably at least 94 pg/mL, most preferably at least 96 pg/mL.

In some embodiments, the inflammatory marker is at least one selected from the group consisting of C-reactive protein (CRP), serum amyloid A, procalcitonin, cytokines, TNFα, and interleukins 6 (IL-6). In a preferred embodiment, the inflammatory marker is IL-6. In some embodiments, the inflammatory marker has a release level of no more than 200 pg/mL, preferably no more than 195 pg/mL, preferably no more than 190 pg/mL, preferably no more than 185 pg/mL, preferably no more than 180 pg/mL, preferably no more than 175 pg/mL, preferably no more than 170 pg/mL, preferably no more than 165 pg/mL, preferably no more than 165 pg/mL, preferably no more than 160 pg/mL, preferably no more than 155 pg/mL, preferably no more than 150 pg/mL, preferably no more than 145 pg/mL, preferably no more than 140 pg/mL, preferably no more than 135 pg/mL, preferably no more than 130 pg/mL, preferably no more than 125 pg/mL, preferably no more than 120 pg/mL, preferably no more than 115 pg/mL, preferably no more than 110 pg/mL, preferably no more than 105 pg/mL, preferably no more than 100 pg/mL, preferably no more than 95 pg/mL, preferably no more than 90 pg/mL, preferably no more than 85 pg/mL, preferably no more than 80 pg/mL, preferably no more than 75 pg/mL, preferably no more than 70 pg/mL, most preferably no more than 65 pg/mL.

In some embodiments, the bioactive dental composite releases at least one of nitrate, potassium, and fluoride on the surface of the tooth. Nitrate and potassium help assuage tooth sensitivity by disrupting nerve signaling and stabilizing nerve function, while fluoride strengthens tooth enamel and supports remineralization. These components boost the tooth's protection, reduce discomfort, and support overall oral health. In some embodiments, the bioactive dental composite has an average potassium release level of at least 500 parts per million (ppm), preferably at least 550 ppm, preferably at least 600 ppm, preferably at least 650 ppm, preferably at least 700 ppm, preferably at least 750 ppm, preferably at least 800 ppm, preferably at least 850 ppm, preferably at least 900 ppm, preferably at least 950 ppm, preferably at least 1000 ppm, preferably at least 1050 ppm, preferably at least 1100 ppm, preferably at least 1150 ppm, preferably at least 1200 ppm, preferably at least 1250 ppm, preferably at least 1300 ppm, preferably at least 1350 ppm, preferably at least 1400 ppm, preferably at least 1450 ppm, most preferably at least 1500 ppm. In some embodiments, the bioactive dental composite has an average nitrate release level of at least 45 ppm, preferably at least 46 ppm, preferably at least 47 ppm, preferably at least 48 ppm, preferably at least 49 ppm, preferably at least 50 ppm, preferably at least 51 ppm, preferably at least 52 ppm, preferably at least 53 ppm, preferably at least 54 ppm, preferably at least 55 ppm, preferably at least 56 ppm, preferably at least 57 ppm, most preferably at least 58 ppm. In some embodiments, the bioactive dental composite has an average fluoride release level of at least 3 ppm, preferably at least 3.2 ppm, preferably at least 3.4 ppm, preferably at least 3.6 ppm, preferably at least 3.8 ppm, preferably at least 4 ppm, preferably at least 4.2 ppm, preferably at least 4.4 ppm, preferably at least 4.6 ppm, preferably at least 4.8 ppm, preferably at least 5 ppm, preferably at least 5.2 ppm, preferably at least 5.4 ppm, preferably at least 5.6 ppm, preferably at least 5.8 ppm, most preferably at least 6 ppm.

### EXAMPLES

The following examples demonstrate a method of vital pulp therapy using bioactive materials as described herein. The examples are provided solely for illustration and are not to be construed as limitations of the present disclosure, as many variations thereof are possible without departing from the spirit and scope of the present disclosure.

### Example 1: Calcium Silicate Cement

White Mineral Trioxide Aggregate (MTA) (ProRoot MTA; Dentsply Tulsa Dental, Tulsa, USA) was used as a state-of-the-art control material and was mixed under aseptic circumstances according to the manufacturer's instructions. Each sample was made as 2 mm thick, 5.5 mm diameter discs to fit into 24 trans-well plates. The discs were placed at 37° C. in a humidified environment with 5% CO₂ and 95% O₂ for 24 hours to ensure the complete setting of the discs.

### Example 2: Potassium Nitrate/Glass Ionomer Cement

The KNO₃ powder was mixed with glass ionomer cement (GIC) powder in two different percentages: 5% and 10%. Then, it was mixed with distilled water, as the liquid phase, in a 2:1 powder-to-liquid ratio and made as 2 mm thick, 5.5 mm diameter discs to fit into 24 trans-well plates. The discs were placed at 37° C. in a humidified environment with 5% CO₂ and 95% O₂ for 24 hours to ensure the setting of the discs. Unmodified GIC prepared in the same manner without adding KNO₃ was used as a control group.

### Example 3: Monocytes Cells Isolation

Using Ficoll-Paque PREMIUM 1.073 separation gradient, human peripheral blood mononuclear cells (PBMC) were extracted from one-day-old buffy coats obtained from six healthy donors at the blood bank in King Fahad University Hospital, Khobar. The buffy coat was first diluted with an equal volume of HBSS, and then 4 ml was layered on top of 3 ml of the Ficoll-Paque PREMIUM 1.073 solution and centrifuged at 400 g for 40 min at 20° C. without a break. After that, the layer of mononuclear cells was transferred to a sterile centrifuge tube and re-suspended and washed in HBSS. Centrifugation was repeated at 500 g for 15 min at 20° C. Subsequently, the supernatant was removed, and the cells were counted using an automated cell counter (Nucleocounter® NC-202TM; Chemometec A/S Allerod, Denmark). The cells were diluted to the required concentration (1,000,000 cells/ml) in complete culture media (RPMI containing 5% FBS, 1% Penicillin-Streptomycin and 1% L-glutamine; Sigma). The cells were seeded in the bottom well of the 24 trans-well plate (1,000,000 cells per well) to receive the different materials in the upper insert.

The peripheral blood mononuclear cells (PBMC) were seeded on the bottom of 24 trans-well plate and received complete culture media RPMI-1640 (Solarbio) media as described above. The cells in each group were cultured with or without LPS 10 Nanograms Per Milliliter (ng/ml) (Escherichia coli serotype, Sigma Aldrich) added to the prepared media. Then, a trans-well chamber was positioned onto each 24 trans-well plate in which the respective material disc was placed. The bottom of the 24 trans-well insert contains micro-pores (0.4 μm) which allowed leaching of ions to the lower compartment of the well where cells are seeded without direct contact between the cells and the material. The PBMC in each group was cultivated in 5 separate wells of a 24 trans-well plate. Table 1 shows the sample number distribution among the groups. All samples were tested on each donor (a total of 5 donors).

TABLE 1
Experimental groups tested based on the
material placed in the trans-well system

| Without LPS stimulation | With LPS stimulation |
|---|---|
| No material (n = 5) | No material (n = 5) |
| MTA material disc (n = 5) | MTA material disc (n = 5) |
| GIC Cement (n = 5) | GIC Cement (n = 5) |
| KNO₃ (5%) + GIC Cement (95%) | KNO₃ (5%) + GIC Cement (95%) |
| (n = 5) | (n = 5) |
| KNO₃ (10%) + GIC Cement (90%) | KNO₃ (10%) + GIC Cement (90%) |
| (n = 5) | (n = 5) |

### Example 4: ELISA for Monocytes Inflammatory Response

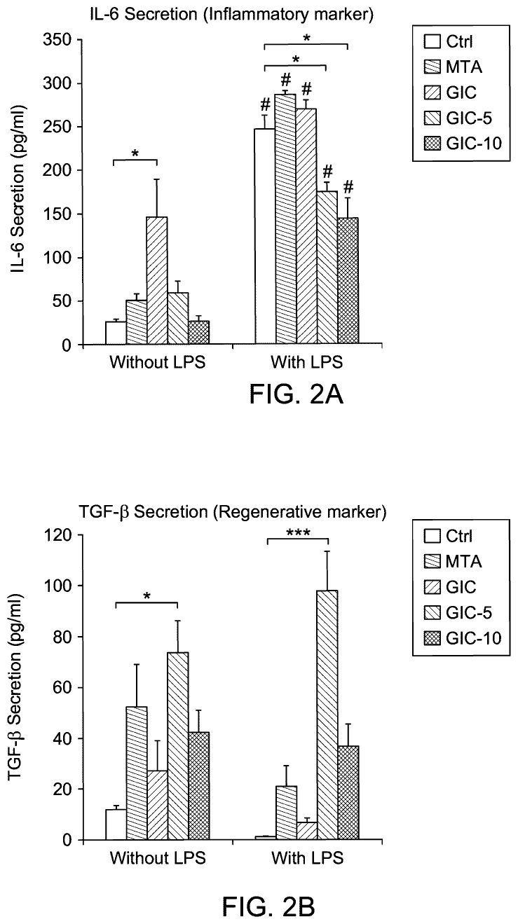

Part of the culture media was transferred to separate tubes, centrifuged for 5 min at 400 g, and then the supernatant was transferred to new tubes. This supernatant was used for enzyme-linked immunosorbent assay (ELISA) in a spectrophotometric plate reader (xMark, BIO-RAD, Microplate Spectrophotometer, USA). Human ELISA kits (MOLEQULE-ON, Auckland, New Zealand) were used, according to the manufacturer's instructions, targeting representative processes related to inflammation (interleukin-6; IL-6) and regeneration (transforming growth factor-beta; TGF-β). The result of enzyme-linked immunosorbent assay (ELISA) is presented in FIGS. 2A and 2B. LPS stimulates cells through Toll-like receptor 4 (TLR4), which causes the release of inflammatory cytokines and the upregulation of costimulatory molecules, thus mimicking the inflammatory response of a tooth having a dental cavity in a patient. Compared to the control group, the GIC group without LPS revealed significantly more IL-6 (p=<0.05). In contrast to the control group, the LPS GIC 5% and GIC 0% groups showed significantly less mean of IL-6 (p=<0.05, p=<0.001). There was a significantly higher amount of TGF-β mean released levels without LPS (p=<0.05). When LPS was present, the GIC 5% group released TGF-β at a significantly higher mean (p=<0.0001) than the control group.

### Example 3: Cell Counting and Viability Assays

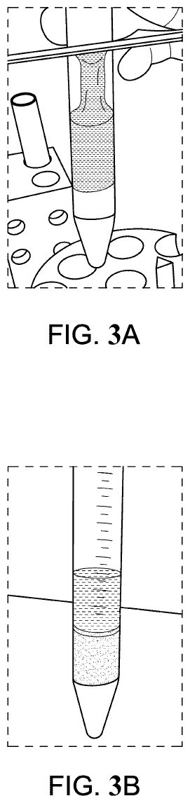

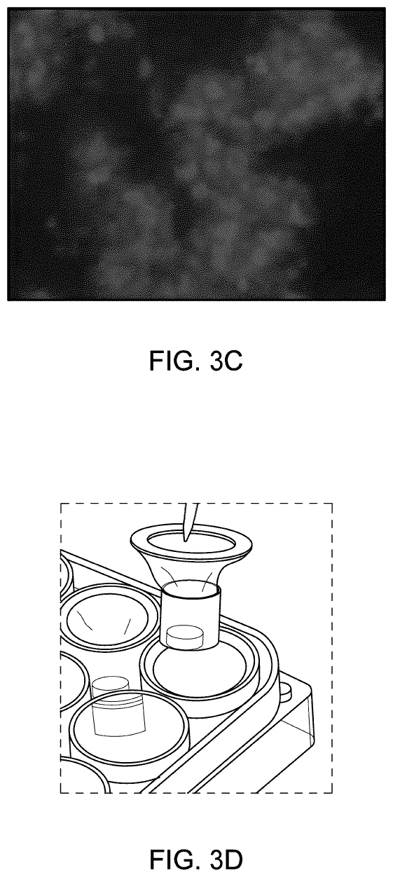

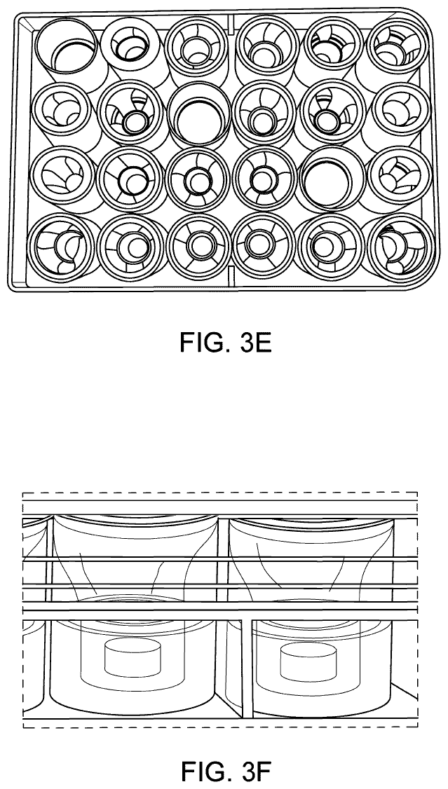

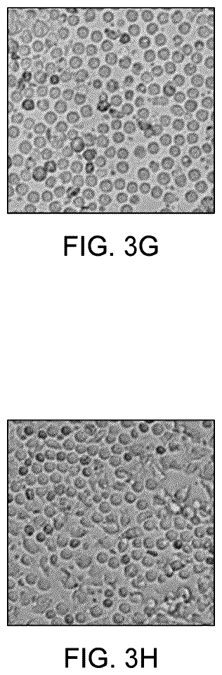

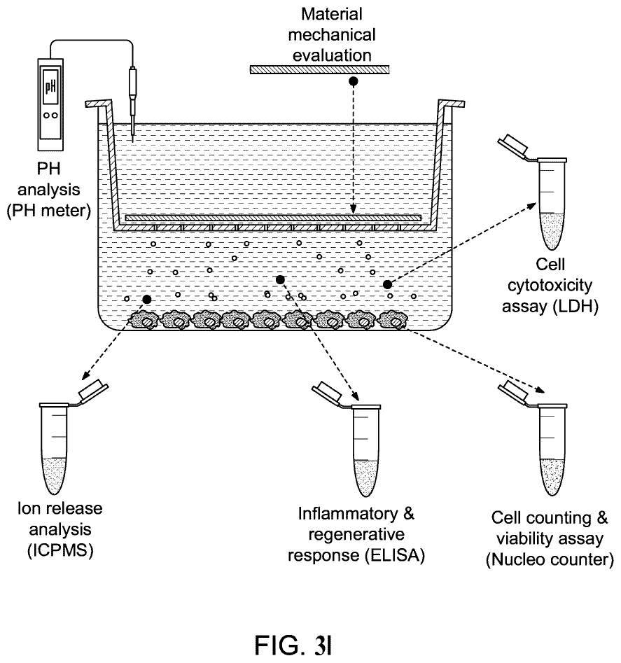

The total and dead cell numbers and viability were determined after 1 day of culture in the NucleoCounter (ChemoMetec). Briefly, the cells on the bottom of the wells were detached using trypsin/EDTA (0.25% trypsin-ethylenediaminetetraacetic acid; Sigma-Aldrich, St. Louis, MO, USA) and centrifuged at 500 g. Thereafter, the cell pellet is dissolved in 1 ml PBS and a special collection device (Via2-Cassette; ChemoMetec) was used to aspirate 60 μL of the cell suspension. The Via2-Cassette is a cell sampling and staining system with acridine orange dye that labels the cell membrane of all cells (dead and live) and DAPI (4′,6-diamidino-2-phenylindole), which stains the nucleus and hence only dead cells with disrupted membrane will be labeled. Cell count data for the different groups (control, MTA, GIC, GIC 5%, GIC 10%) is presented in FIGS. 3A to 3I. Cell counting results showed that groups without lipopolysaccharide (LPS) differed considerably from the control group, with the GIC 10% group having a significantly lower percentage (p=<0.0001). When compared to the control group, the GIC 10% group had significantly fewer viable cells (p=<0.0001). Regarding cell attachment, both the attached and detached GIC 10% group showed significantly less than the control group (p=<0.0001). Although GIC 5% result was not significantly changed from the control group (p=>0.05) but there were significant changes between GIC 5% groups with/without LPS.

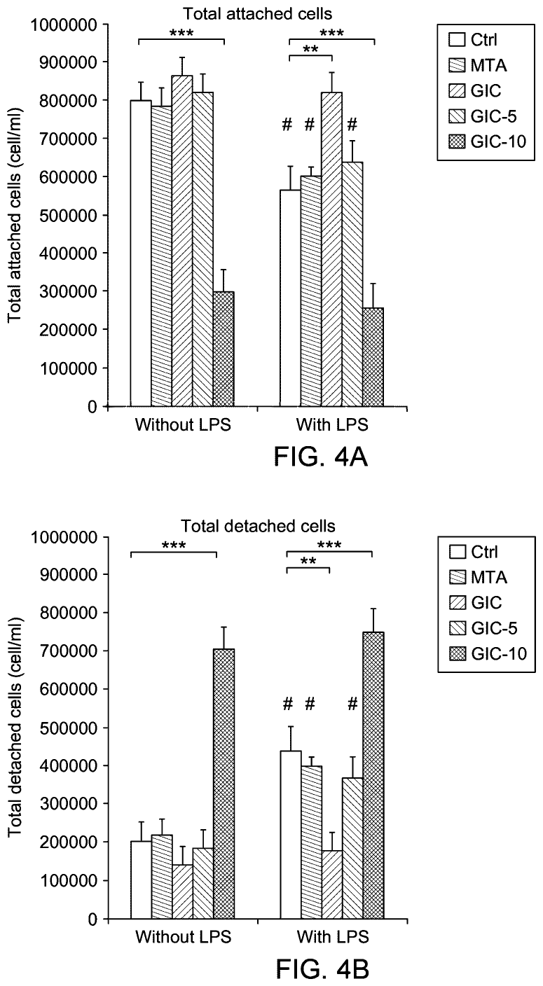

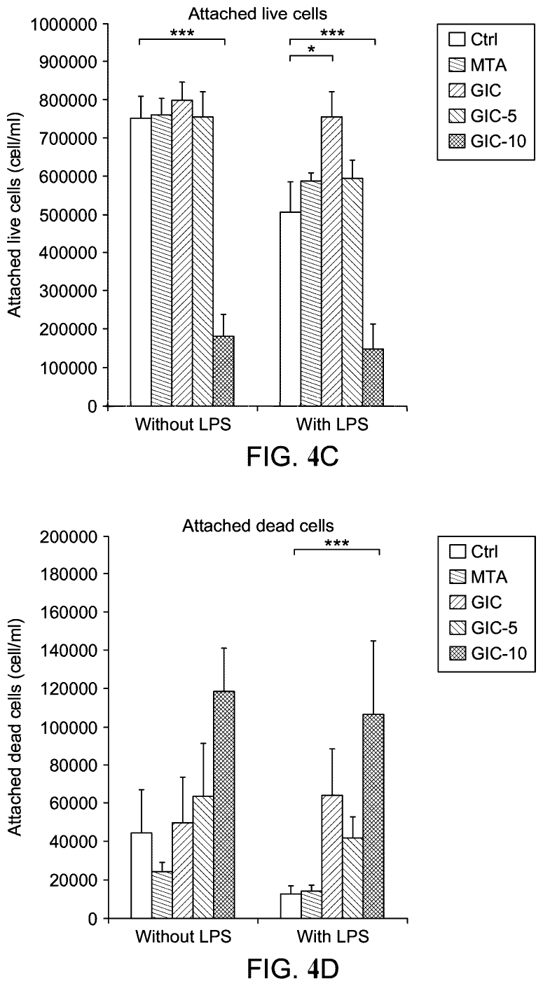

In addition, the cell counting results showed that there were also significant differences between the groups after LPS stimulation. The GIC 10% group was significantly lower than the control group (p=<0.0001). In addition, there were significantly fewer viable cells in the GIC 10% group (p=<0.001) than with the control group. Conversely, the GIC group outperformed the control group significantly (p=<0.05). Additionally, the GIC 10% group's total attached and detached cells were considerably lower than those of the control group (p=<0.05). Regardless of whether they are attached or not, the GIC group had a significantly higher (p=<0.05) correlation. The result is presented in (FIGS. 4A to 4D). The results observed for the GIC 5% group did not exhibit a statistically significant difference compared to the control group (p≥0.05). However, notable differences were identified between the GIC 5% subgroup that was administered LPS and the GIC 5% subgroup that was not administered LPS.

### Example 5: PH Changes Test

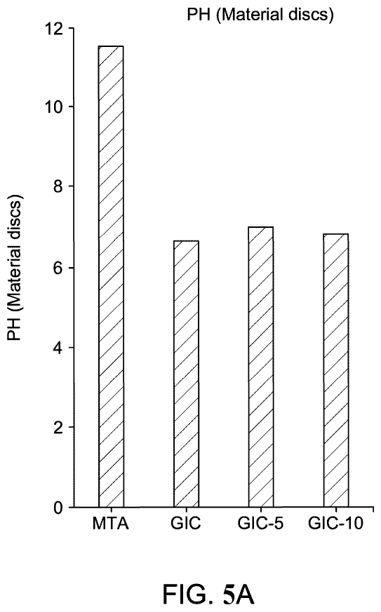

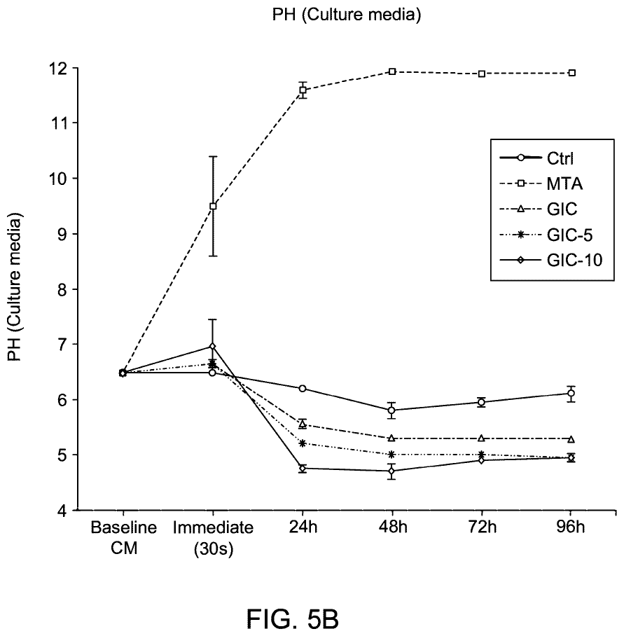

Samples from each group were submerged in 10 mm culture media to simulate the cell experiment. The pH changes over time were evaluated before placing discs and then at 30 seconds, 24, 48, 72, and 96 hours using a pH meter (Thermo Scientific™ Orion™ Benchtop PH Meters). FIG. 5A illustrates that the disc pH after a 24-hour preparation period after mixing is strongly alkaline for MTA (11.4), while nearly neutral pH values were found for GIC, GIC 5%, and GIC 10% (6.6, 6.9, 6.7) respectively. Ten discs from each group were submerged in 10 mm of culture media to replicate the cellular experiment, with two samples from each group for pH measurement using culture media. The culture media's mean pH was measured (6.5) before the discs were placed, and 30 seconds later, the mean pH of the following groups-control, MTA, GIC, GIC 5%, and GIC 10% was measured (6.5, 9.5, 6.5, 6.5, 6.9), respectively. After that, pH was measured for 24, 48, 72, and 96 hours. After 24 hours, the mean pH was, in that order, (6.2, 11.6, 5.5, 5.2, 4.7). The mean pH was 5.8, 11.9, 5.3, 5.0, and 4.7 after 48 hours, respectively. At 72 and 96 hours, respectively, the mean pH of MTA, GIC, and GIC 10% remained constant at 11.9, 5.3, and 4.9. GIC 5% slightly changed (5.0, 4.9) at 72 hours and 96 Hours as presented in (FIG. 5B).

### Example 6: Ion Release Analysis

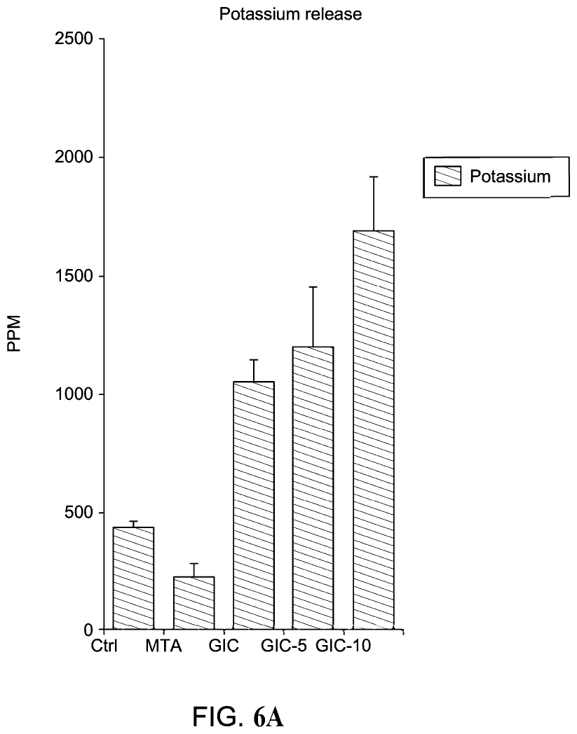

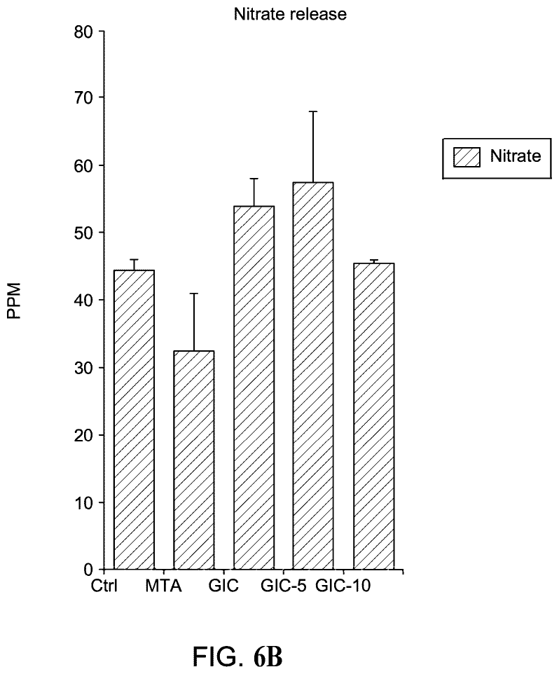

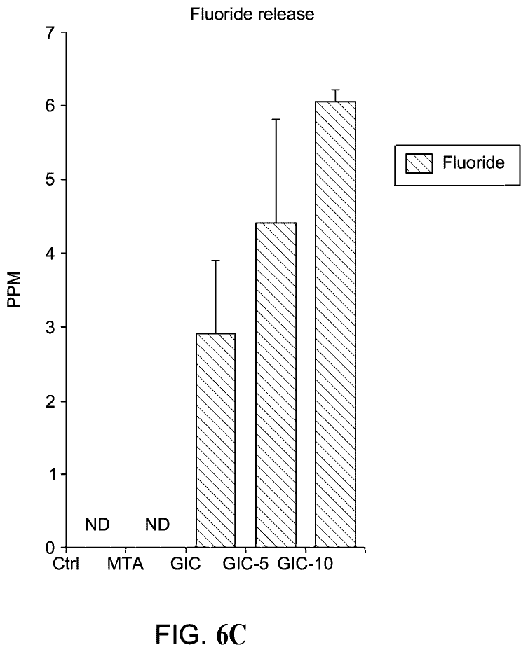

To replicate the biological experiment, 10 discs from each group were submerged in 10 mm of culture media, with two samples per group. The culture media was gathered and examined 1 day after the disc submersion using an inductively Coupled Plasma Mass Spectroscopy (ICP-MS) analytical technique to measure selected elements within the sample matrix based on their ionization. The mean potassium release levels were 443, 220, 1046, 1198, and 1683 in the control, MTA, GIC, GIC 5%, and GIC 10%, respectively. In contrast, the mean nitrate level was (44, 32, 54, 57, 45) in that order. FIGS. 6A to 6C shows the measured fluoride release amounts for GIC, GIC 5%, and GIC 10% (2.9, 4.4, and 6). The rises in KNO₃ were correlated with an increase in fluoride emission.

### Example 7: Compression Test

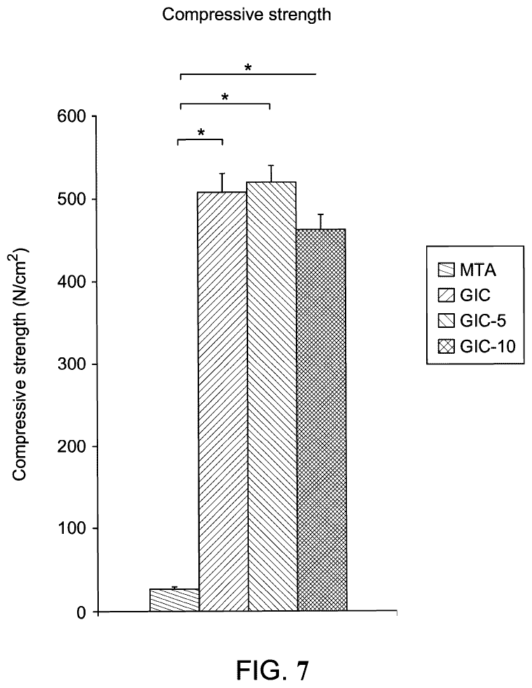

Compressive strength (CS) was measured using a universal testing machine (5569A, Instron, UK) fitted with a flat crosshead and a load cell (50 kN capacity). Specimens were placed on stainless steel base during the test and the extension speed of the crosshead was 1 mm/min. As presented in FIG. 7, the compressive strength test results indicate that the MTA group exhibits significantly less strength than the GIC, GIC 5%, and GIC 10% groups (p=<0.0001*, p=<0.0001, and p<0.0001).

### Example 8: Microhardness Test

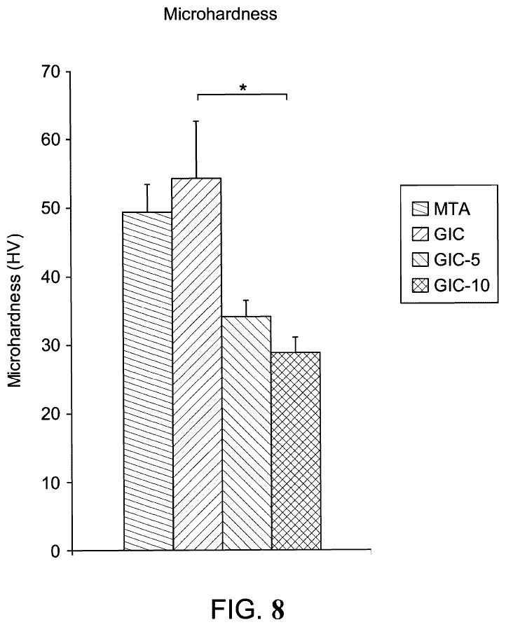

Test specimens were taken to the microhardness tester FM-700 (Future-Tech Corp. Tokyo, Japan), equipped with a Vickers diamond that was applied on the surface with a load of 50 g/30 s. As presented in FIG. 8, the GIC 10% group has a significantly higher (p=<0.05) Vickers microhardness than the MTA group. Additionally, the GIC group outperformed the GIC 5% and GIC 10% groups significantly (p=<0.05 and p=<0.01), respectively.

### Example 9: Cytotoxicity

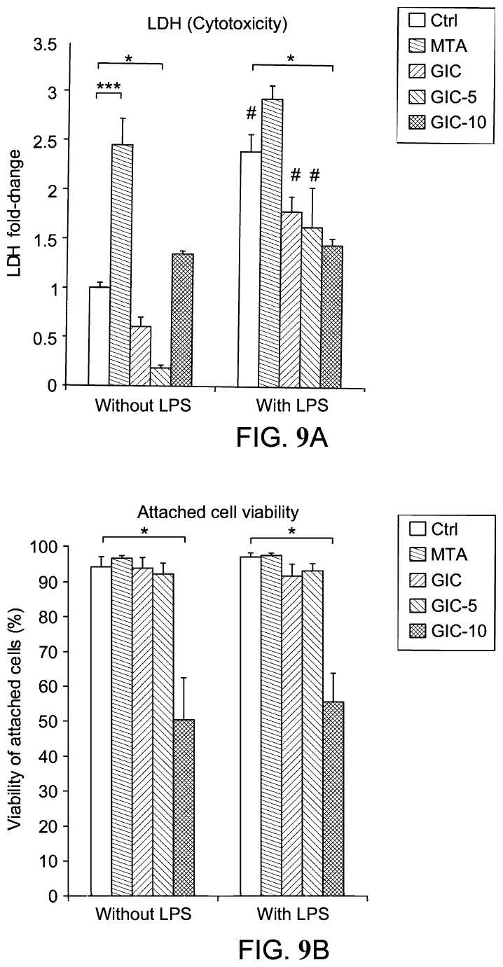

Cytotoxicity measurement is presented in FIGS. 9A and 9B. Compared to the control group, the MTA group had significantly greater LDH levels (higher cytotoxicity) (p=<0.0001) without LPS. In contrast, the GIC 5% group was significantly lower (p=<0.005) compared to the control group. In contrast to the control group, GIC 10% was significantly lower (p=<0.05) in the presence of LPS. The other groups showed no statistical significance. The control, GIC, and GIC 5% changed significantly after LPS exposure. Additionally, the result of attached live cell groups didn't change with and without LPS.

The data was analyzed using SPSS package version 28.0.1 (SPSS Inc.). A two-way ANOVA was conducted, considering material type as the primary independent variable and lipopolysaccharide LPS stimulation as the secondary variable. Bonferroni's test was followed by correction for post hoc comparisons. Statistical significance was set at p<0.05. The data are presented in the graphs as means accompanied by the standard error of the mean.

Numerous modifications and variations of the present disclosure are possible in light of the above teachings. It is therefore to be understood that within the scope of the appended claims, the invention may be practiced otherwise than as specifically described herein.

1. A method of vital pulp therapy, comprising:
drilling into a pulp of a tooth to form a hole, then excavating infected pulp from the hole, wherein the tooth has a cavity breaching at least a surface of the pulp and is under lipopolysaccharide (LPS) stimulation during the drilling;
mixing a glass ionomer cement (GIC) and potassium nitrate (KNO₃) with a solvent to form a bioactive capping material; and
covering remaining healthy pulp in the hole of the tooth with the bioactive capping material and curing the bioactive capping material to form a cured capping material,
wherein the KNO₃ is present in the bioactive capping material in an amount of 1 to 10 wt. % based on the weight of the bioactive capping material, and
wherein after the curing, the patient has an IL-6 secretion that is 40 to 75% less than the IL-6 secretion after curing with a substantially identical method using the same bioactive capping material without the KNO₃.

2. The method of claim 1, wherein a ratio of the solvent to a combined amount of GIC and KNO₃ is 1:3 to 3:1.

3. The method of claim 1, wherein the solvent is selected from the group consisting of water, ethanol, methanol, and acetone.

4. The method of claim 1, wherein the solvent is water.

5. The method of claim 1, wherein the cured capping material has a compressive strength of at least 450 N/cm².

6. The method of claim 1, wherein the cured capping material has a microhardness of no more than 40 HV.

7. The method of claim 1, wherein the KNO₃ is present in an amount of 5 to 10 wt. % based on the weight of the capping material.

8. The method of claim 1, wherein the bioactive capping material has a pH of 6 to 7.

9. The method of claim 1, wherein the cured capping material has a compressive strength of at least 500 N/cm².

10. The method of claim 1, wherein the cured capping material has a microhardness of no more than 35 HV.

11. The method of claim 1, wherein the bioactive capping material has a pH of 6.5.

12. A method of treating inflammation near a tooth comprising:
drilling into a pulp of a tooth of a patient to form a hole, then excavating infected pulp from the hole, wherein the tooth has a cavity breaching at least a surface of the pulp; and
administering a bioactive dental composite comprising a glass ionomer cement (GIC) and potassium nitrate to at least one surface of remaining healthy pulp of the tooth of a patient,
wherein the potassium nitrate is present in an amount of 1 to 10 wt. % based on the weight of the dental composite, and
wherein the dental composite promotes the release of a regenerative marker and inhibits the release of an inflammatory marker.

13. The method of claim 12, wherein the regenerative marker has a release level of at least 40 pg/mL.

14. The method of claim 12, wherein the regenerative marker is at least one selected from the group consisting of growth differentiation factor (GDF), inhibin A (INHA), myostatin (MSTN), and transforming growth factor (TGF-β).

15. The method of claim 12, wherein the inflammatory marker has a release level of at least 25 pg/mL.

16. The method of claim 12, wherein the inflammatory marker is at least one selected from the group consisting of C-reactive protein (CRP), serum amyloid A, procalcitonin, cytokines, TNFα, and interleukins 6 (IL-6).

17. The method of claim 12, wherein the bioactive dental composite releases at least one of nitrate, potassium, and fluoride on the surface of the tooth.

18. The method of claim 12, wherein the bioactive dental composite has an average potassium release level of at least 1000 ppm.

19. The method of claim 12, wherein the bioactive dental composite has an average nitrate release level of at least 50 ppm and an average fluoride release level of at least 4 ppm.

20. The method of claim 1, wherein LPS stimulation comprises injecting a lipopolysaccharide into an area surrounding the tooth to activate an immune response and induce inflammatory cytokine production.
